(19) United States
(12) Patent Application Publication
(10) Pub. No.: US 2026/0182866 A1
(43) Pub. Date: Jul. 2, 2026

(54) INTEGRATED IN VIVO SYSTEM FOR DISEASE DETECTION, DIAGNOSIS, AND TARGETED THERAPEUTIC DELIVERY

(71) Applicant: Novelna Inc., Palo Alto, CA (US)

(72) Inventor: Ashkan AFSHIN, Santa Clara, CA (US)

(73) Assignee: Novelna Inc., Palo Alto, CA (US)

(21) Appl. No.: 19/429,854

(22) Filed: Dec. 22, 2025

Related U.S. Application Data
(60) Provisional application No. 63/739,933, filed on Dec. 30, 2024.

(51) Int. Cl. A61B 5/145; A61B 5/00; G16H 50/20

(52) U.S. Cl. CPC A61B 5/145; A61B 5/4839; G16H 50/20

(57) ABSTRACT

A method for in vivo detection, diagnosis, and therapeutic intervention of diseases is disclosed. The method includes utilizing synthetic sensors configured to interact with disease-specific signals, wherein the signals are selected from the group consisting of proteins, post-translational modifications (PTMs), RNA, DNA, metabolites, or combinations thereof; employing a diagnostic kit designed to collect and process signals excreted or released into body fluids, wherein the body fluids are selected from the group consisting of urine, saliva, sweat, tears, exhaled breath, or combinations thereof; and analyzing the collected signals using a reader configured to process the signals and integrate the data with an artificial intelligence (AI) platform to generate real-time diagnostic outputs.

### CROSS-REFERENCE TO RELATED APPLICATIONS

This application claims priority to U.S. Provisional Ser. No. 63/739,933 filed on Dec. 30, 2024, which is incorporated herein by reference in its entirety for any and all purposes.

### TECHNICAL FIELD

This application relates generally to integrated in vivo system for disease detection, diagnosis, and targeted therapeutic delivery.

### BACKGROUND

Early detection of diseases is critical for improving patient outcomes and reducing healthcare costs. Aberrant molecular changes, including post-translational modifications (PTMs), gene expression, RNA signatures, protein alterations, and metabolite profiles, are hallmarks of many diseases and play a pivotal role in their initiation, progression, and transformation. Advances in mass spectrometry (MS), genomics, metabolomics, and artificial intelligence (AI) have enabled the discovery of tissue-specific molecular fingerprints that can serve as sensitive and specific biomarkers for early disease detection. Current diagnostic methods often require invasive procedures or external sample processing, which limits their accessibility and scalability. Furthermore, these techniques are largely focused on specific sample types, such as blood, which may not always provide sufficient biomarker diversity. To address these challenges, there is a growing need for diagnostic systems that can analyze a broader range of body fluids, including urine, saliva, sweat, tears, and exhaled breath. These body fluids offer non-invasive or minimally invasive alternatives that can provide critical diagnostic insights while enhancing patient comfort and compliance.

### SUMMARY

The present invention provides a method for in vivo detection of diseases through the identification of tissue-specific molecular fingerprints. The system consists of three primary components:

Synthetic Sensor: A sensor that interacts with disease-specific biomarkers in the tissue microenvironment. The synthetic sensor can take various forms, including peptides, synthetic peptides, small molecules, molecular glue, or hybrid combinations, tailored to bind specifically to targeted biomarkers. In some embodiments, the sensor also serves therapeutic purposes, delivering agents or functioning as a therapeutic itself.

Body Fluid-Based Diagnostic Kit: A non-invasive diagnostic kit designed to capture and process signals from the synthetic sensor. Signals from the synthetic sensor are excreted or released into body fluids such as urine, saliva, sweat, tears, or exhaled breath, enabling collection and subsequent analysis. This adaptability ensures that the system is applicable to a wide range of diseases and patient scenarios.

Reader: A portable or wearable device that detects and analyzes signals generated by the interaction between the synthetic sensor and the biomarkers. The reader processes the data and integrates it with an AI platform for real-time diagnostic insights, delivering accurate results that can guide clinical decisions.

The invention is designed to be non-invasive, scalable, and adaptable for detecting a wide range of diseases. It integrates advanced AI, multi-omics-based biomarker discovery, and innovative detection mechanisms to ensure precise and timely diagnosis using a variety of body fluids.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification. Aspects can be combined, and it will be readily appreciated that features described in the context of one aspect of the invention can be combined with other aspects. Aspects can be implemented in any convenient form, for example, by appropriate computer programs, which may be carried on appropriate carrier media (computer readable media), which may be tangible carrier media (e.g., disks) or intangible carrier media (e.g., communications signals). Aspects may also be implemented using any suitable apparatus, which may take the form of programmable computers running computer programs arranged to implement the aspect. As used in the specification and in the claims, the singular form of ‘a,’ ‘an,’ and ‘the’ include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing.

FIG. 1. The approach includes disease-specific biomarker target discovery, synthetic sensor development, patient self-administration, and data analysis and transfer of results to EHR and physician.

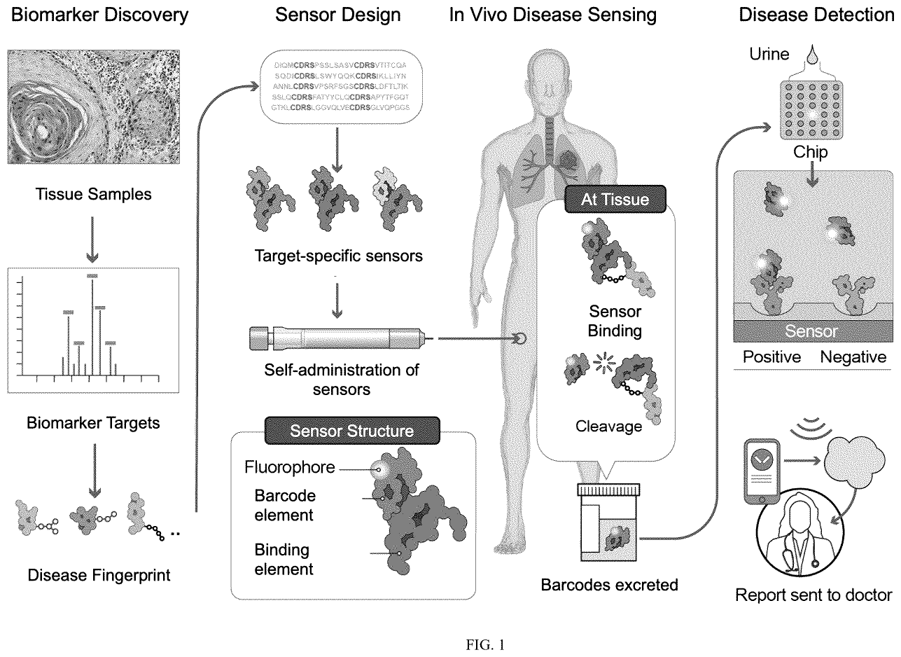

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

### Biomarker Type

Detection of Protein Biomarkers. In one embodiment, the synthetic sensor is specifically configured to detect protein biomarkers that are uniquely expressed in diseased tissues or exhibit alterations in concentration when compared to levels observed in healthy tissues.

Detection of Post-Translational Modifications (PTMs). In another embodiment, the synthetic sensor is adapted to detect post-translational modifications, including but not limited to glycosylation and phosphorylation, which serve as indicators of disease progression and tissue-specific abnormalities.

Detection of RNA Biomarkers. In a further embodiment, the synthetic sensor is designed to bind to RNA biomarkers expressed within diseased tissues. Upon binding, the sensor facilitates the release of a detectable signal, enabling subsequent analysis.

Detection of Genetic Mutations or Variants. In yet another embodiment, the synthetic sensor interacts with genetic mutations or DNA variants associated with specific diseases. These interactions result in the generation of signals that are excreted into urine and are subsequently analyzed by the system.

Detection of Metabolite Biomarkers. In another embodiment, the synthetic sensor is configured to detect metabolites indicative of disease-specific metabolic dysregulation. These disease-associated metabolites produce measurable signals that are processed for diagnostic purposes.

Detection of Combination Biomarkers. In a further embodiment, the synthetic sensor is capable of detecting combinations of multiple biomarker types, including proteins, PTMs, RNA, DNA, and metabolites. This embodiment is designed to enhance diagnostic accuracy by capturing a comprehensive molecular fingerprint of the disease state.

Detection of Customizable Biomarker Panels. In certain embodiments, the system is tailored to detect biomarker panels that are specifically selected based on the pathology being targeted. This customization allows for precise and disease-specific diagnostic applications.

### Sensor Types

Peptide-Based Sensors. In one embodiment, the synthetic sensor is composed of natural peptides that are specifically engineered to bind to targeted disease biomarkers with high specificity. For example, peptide-based sensors may detect overexpressed proteins in cancer cells and trigger the generation of a diagnostic signal.

Synthetic Peptide-Based Sensors. In another embodiment, the synthetic sensor comprises engineered peptides that exhibit enhanced stability and increased binding affinity for specific disease biomarkers. For example, synthetic peptides may be designed to bind glycosylated proteins associated with diabetes, providing precise detection capabilities.

Small Molecule-Based Sensors. In a further embodiment, the synthetic sensor is formulated using chemical compounds that are specifically designed to selectively bind to disease-specific biomarkers and produce a measurable diagnostic signal. For example, small molecules may target key metabolic enzymes in cancer cells, enabling the detection of tumor activity with high accuracy.

Molecular Glue-Based Sensors. In yet another embodiment, the synthetic sensor utilizes chemical agents referred to as molecular glue, which facilitate or stabilize interactions between the disease biomarkers and the sensor, thereby enhancing detection efficiency. For example, molecular glue may be used to bridge mutated DNA fragments to the sensor, enabling efficient detection of genetic mutations.

Hybrid Sensors. In an additional embodiment, the synthetic sensor combines multiple sensor types, including peptides, synthetic peptides, small molecules, and molecular glue, to achieve improved diagnostic sensitivity and specificity. For example, a hybrid sensor may employ synthetic peptides to detect protein biomarkers and molecular glue to identify DNA mutations, enabling simultaneous multi-omic analysis for comprehensive disease characterization.

### Therapeutic Applications of the Sensor

Sensor as a Delivery Mechanism. In one embodiment, the synthetic sensor is engineered to function as a delivery vehicle for therapeutic agents, specifically targeting diseased tissues. For example, the sensor may deliver therapeutic agents such as chemotherapeutics directly to the tumor microenvironment. The therapeutic agent may be conjugated to the synthetic sensor, with the release of the agent occurring upon binding of the sensor to the target biomarker.

Sensor as a Therapeutic Agent. In another embodiment, the synthetic sensor itself is configured to act as a therapeutic agent. For instance, the sensor may be designed as an antibody or an antibody-like structure capable of binding to and neutralizing specific cancer cells or other diseased cells. The sensor may induce immune-mediated cytotoxicity against the target cells or disrupt critical molecular pathways essential for disease progression.

Treatment of Metastatic Disease. In a further embodiment, the synthetic sensor is engineered to facilitate the treatment of metastatic disease by delivering therapeutic agents to secondary tumor sites or other affected tissues. The sensor may be designed to recognize metastatic biomarkers and carry therapeutic agents to these sites, thereby enabling targeted intervention for metastases. Additionally, the synthetic sensor itself may serve as a therapeutic by specifically targeting and neutralizing metastatic cancer cells.

Combination Therapeutic and Diagnostic Functionality. In another embodiment, the synthetic sensor is configured to perform both therapeutic and diagnostic functions. The sensor may simultaneously identify disease-specific biomarkers and deliver targeted therapy to the affected tissues, thus providing integrated diagnostic and therapeutic capabilities.

### Applications for Specific Disease Categories

Detection of a Single Disease. In one embodiment, the system is specifically configured to detect biomarkers unique to a single disease. This embodiment may include, but is not limited to:

Neurological Disorders and Neurodegenerative Diseases: Alzheimer's disease, Parkinson's disease, amyotrophic lateral sclerosis (ALS), multiple sclerosis (MS), epilepsy, stroke, frontotemporal dementia, and other neurodegenerative or neurological conditions characterized by specific biomarker profiles.

Cancers: Solid tumors and hematologic malignancies such as breast cancer, lung cancer (including small cell and non-small cell), colorectal cancer, pancreatic cancer, prostate cancer, ovarian cancer, cervical cancer, endometrial cancer, gastric cancer, esophageal cancer, liver cancer, kidney cancer, bladder cancer, thyroid cancer, melanoma, glioblastoma, lymphoma (Hodgkin and non-Hodgkin), leukemia, and multiple myeloma.

Other Diseases: Chronic obstructive pulmonary disease (COPD), diabetes mellitus (type 1 and type 2), cardiovascular diseases (e.g., atherosclerosis, myocardial infarction, heart failure), autoimmune diseases (e.g., systemic lupus erythematosus, rheumatoid arthritis, inflammatory bowel disease), and infectious diseases (e.g., tuberculosis, hepatitis, and HIV).

Detection of Related Diseases. In another embodiment, the system is adapted to detect biomarkers associated with a group of related diseases. For example, the system may identify biomarkers pertinent to: multiple cancer types, including epithelial, mesenchymal, and hematological malignancies; and neurological disorders with overlapping pathophysiological mechanisms, such as Alzheimer's disease, Parkinson's disease, and Lewy body dementia.

Detection of Organ-Specific Diseases. In a further embodiment, the system is designed to diagnose diseases affecting a single organ system. Examples include:
Pulmonary Conditions: Chronic obstructive pulmonary disease (COPD), asthma, pulmonary fibrosis, and lung cancer.
Gastrointestinal Disorders: Crohn's disease, ulcerative colitis, irritable bowel syndrome (IBS), gastrointestinal cancers, and celiac disease.
Hepatic Diseases: Non-alcoholic fatty liver disease (NAFLD), cirrhosis, hepatocellular carcinoma, and hepatitis.

Detection of Combinations of Diseases. In an additional embodiment, the system is configured to simultaneously detect biomarkers for multiple diseases within a combination of categories, including single diseases, related diseases, and diseases of the same organ system. For instance, the system may monitor biomarkers for lung cancer, COPD, and pulmonary fibrosis concurrently, providing a comprehensive diagnostic solution for respiratory-related health conditions.

### Patient Groups and Use Cases

Routine Screening. In one embodiment, the system is utilized for routine screening of asymptomatic individuals to detect potential early disease biomarkers. This application is designed to identify diseases at an early stage before the onset of clinical symptoms.

Asymptomatic Individuals at Risk. In another embodiment, the system targets asymptomatic individuals who are at increased risk of developing diseases due to genetic predispositions, lifestyle factors, or environmental exposures. This use case is aimed at proactive health monitoring and risk mitigation.

Individuals with Non-Specific Symptoms. In a further embodiment, the system is applied to diagnose underlying diseases in patients presenting with vague or non-specific symptoms. This application is intended to uncover potential pathological conditions that may otherwise remain undiagnosed.

Symptomatic Patients. In another embodiment, the system is configured to confirm the presence of a disease in patients exhibiting specific clinical symptoms. This use case facilitates accurate and timely diagnoses, enabling appropriate medical interventions.

Patients with Diagnosed Conditions. In yet another embodiment, the system is employed to monitor disease progression, evaluate the effectiveness of therapeutic interventions, and assess remission status in patients with already diagnosed conditions. This application supports long-term disease management and therapeutic optimization.

Advanced Indications. In a further embodiment, the system is used to guide therapeutic decisions, assess disease severity, detect potential relapses, and provide comprehensive insights into the patient's health status. This application is particularly relevant for chronic and complex diseases.

### Medium-specific Applications

In one embodiment, the system is configured to use urine exclusively as the medium for signal detection.

In another embodiment, the system is configured to use tears exclusively as the medium.

In a further embodiment, the system is designed to utilize saliva exclusively as the medium.

In yet another embodiment, the system employs sweat exclusively as the medium.

Additionally, the system can use exhaled breath exclusively for detecting the signal. Combination of Body Fluids.

In one embodiment, the system analyzes a combination of urine and saliva to integrate biomarkers from two distinct physiological sources, enhancing diagnostic accuracy.

In another embodiment, the system utilizes urine and breath in tandem for signal detection.

In a further embodiment, the system processes signals from urine and sweat for signal detection.

### Hybrid Applications

In other embodiments, the system integrates multiple body fluids into a unified diagnostic process.

### Methods of Administration of Sensors

Oral Administration (Ingestion). In one embodiment, the synthetic sensor is formulated for oral administration. The sensor is designed to withstand the acidic environment of the stomach and is absorbed into the systemic circulation via the gastrointestinal tract.

Subcutaneous Administration. In another embodiment, the synthetic sensor is administered subcutaneously, allowing gradual release into the bloodstream or local tissue environment.

Intramuscular Administration (IM). In a further embodiment, the synthetic sensor is delivered via intramuscular injection.

Combination of Methods. In an additional embodiment, the synthetic sensor is administered using a combination of methods tailored to the specific disease or diagnostic requirement.

Customized Delivery Protocols. In certain embodiments, the method of administration is customized based on patient-specific factors, such as age, weight, and the nature of the disease.

### The Number of Sensors Required per Disease

Single Sensor Embodiment. In one embodiment, a single synthetic sensor is utilized to detect a specific biomarker associated with a particular disease. This approach is ideal for diseases where a single biomarker provides sufficient diagnostic specificity and sensitivity.

More Than One Sensor Embodiment. In another embodiment, more than one sensor is deployed to detect multiple biomarkers for a given disease. This embodiment enhances diagnostic accuracy by targeting different aspects of the disease's molecular profile.

More Than Two Sensors Embodiment. In a further embodiment, the system uses more than two synthetic sensors to identify combinations of biomarkers. This configuration is particularly beneficial for complex diseases with multi-dimensional biomarker signatures, such as cancers or autoimmune disorders.

More Than Five Sensors Embodiment. In yet another embodiment, more than five sensors are employed for diseases requiring comprehensive profiling of molecular changes. This embodiment allows simultaneous detection of protein biomarkers, PTMs, metabolites, RNA, and genetic mutations, ensuring a robust and detailed diagnostic output.

More Than Ten Sensors Embodiment. In an additional embodiment, the system incorporates more than ten sensors to detect an extensive array of biomarkers. This embodiment is designed for diseases with highly heterogeneous presentations or for multiplexed diagnostics covering multiple disease states or subtypes.

Dynamic Sensor Allocation Embodiment. In some embodiments, the number of sensors is dynamically determined based on the disease being evaluated and the complexity of its biomarker profile. For example:
A single sensor for early-stage disease detection with high specificity.
Five or more sensors for diseases requiring differential diagnosis or monitoring multiple disease states.
Ten or more sensors for comprehensive multi-omic analyses across heterogeneous disease conditions.

### Signal Detection Methods

Immunoassay-Based Signal Detection. In one embodiment, the system utilizes immunoassays, such as enzyme-linked immunosorbent assays (ELISA) or chemiluminescence-based assays, to detect specific signals with high sensitivity and specificity in body fluids or exhaled breath. Sandwich immunoassays may also be employed for multiplexed detection of multiple signals simultaneously.

Fluorescence-Based Signal Detection. In another embodiment, the system employs fluorescence-based methods, using fluorescent-tagged probes or sensors to identify the presence or concentration of signals. Techniques such as time-resolved fluorescence and Förster resonance energy transfer (FRET) are used for real-time analysis of interactions between the synthetic sensor and target signals.

Mass Spectrometry (MS) for Signal Detection. In a further embodiment, the system integrates high-resolution mass spectrometry methods, including liquid chromatography-mass spectrometry (LC-MS) and tandem mass spectrometry (MS/MS), for precise quantification and characterization of signals. Targeted proteomics techniques, such as selected reaction monitoring (SRM) or parallel reaction monitoring (PRM), are employed for in-depth signal analysis.

Electrochemical Signal Detection. In yet another embodiment, the system uses electrochemical methods to detect signals through changes in conductivity, current, or potential. This approach involves biosensors designed to interact with specific signals, generating measurable electrochemical responses.

Nanoparticle-Based Signal Detection. In another embodiment, the system incorporates nanoparticle-based methods, where functionalized gold or quantum dot nanoparticles are used to detect signals through optical or plasmonic changes, enabling enhanced sensitivity and specificity.

Isothermal Amplification for Signal Detection. In a further embodiment, the system applies isothermal amplification techniques, such as loop-mediated isothermal amplification (LAMP), to detect nucleic acid-based signals (RNA or DNA) present in body fluids.

Microfluidic Signal Detection. In another embodiment, the system integrates microfluidic or lab-on-a-chip devices to automate the processes of sample handling, signal detection, and data analysis. These compact devices enhance efficiency and accuracy in detecting signals in small volumes of body fluids.

Combination of Signal Detection Methods. In an additional embodiment, the system combines multiple detection methods to optimize diagnostic performance. For example, fluorescence-based detection can be paired with microfluidic platforms.

Customized Signal Detection Based on Disease and Fluid Type. In yet another embodiment, the system customizes detection methods based on the disease being diagnosed and the body fluid being analyzed.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A method for in vivo detection, diagnosis, and therapeutic intervention of diseases, comprising:
a. utilizing synthetic sensors configured to interact with disease-specific signals, wherein the signals are selected from the group consisting of proteins, post-translational modifications (PTMs), RNA, DNA, metabolites, or combinations thereof;
b. employing a diagnostic kit designed to collect and process signals excreted or released into body fluids, wherein the body fluids are selected from the group consisting of urine, saliva, sweat, tears, exhaled breath, or combinations thereof; and
c. analyzing the collected signals using a reader configured to process the signals and integrate the data with an artificial intelligence (AI) platform to generate real-time diagnostic outputs.

2. The method of claim 1, wherein the synthetic sensors are selected from the group consisting of natural peptides, synthetic peptides, small molecules, molecular glue, or hybrid combinations thereof.

3. The method of claim 1, wherein the diagnostic kit is specifically configured for individual body fluids, including urine, saliva, sweat, tears, and exhaled breath, and employs signal detection methods comprising:
a. immunoassays, including enzyme-linked immunosorbent assays (ELISA) and chemiluminescence-based assays;
b. fluorescence-based methods, including Förster resonance energy transfer (FRET) and time-resolved fluorescence;
c. mass spectrometry, including liquid chromatography-mass spectrometry (LC-MS) and tandem mass spectrometry (MS/MS);
d. electrochemical detection, including conductivity and potential-based sensors;
e. nanoparticle-based detection using functionalized nanoparticles or quantum dots; and
f. microfluidic platforms for automated detection and analysis.

4. The method of claim 1, wherein the synthetic sensors are further configured to perform therapeutic functions, including:
a. delivery of therapeutic agents directly to diseased tissues;
b. acting as therapeutic agents by neutralizing or targeting diseased cells, including cancer cells; and
c. inducing immune-mediated cytotoxicity or disrupting disease-related pathways.

5. The method of claim 1, wherein the system detects one or more signals, wherein the signals include:
a. a single signal for highly specific disease detection; and
b. a combination of signals comprising proteins, PTMs, RNA, DNA, and metabolites for multi-omic analysis and enhanced diagnostic accuracy.

6. The method of claim 1, wherein the synthetic sensors are administered via methods comprising:
a. oral ingestion;
b. subcutaneous injection;
c. intramuscular injection; and
d. a combination of the above methods tailored to specific diagnostic or therapeutic applications.

7. The method of claim 1, wherein the number of synthetic sensors utilized is dynamically determined based on the disease profile, comprising:
a. a single sensor for detecting specific signals associated with a single disease;
b. more than one sensor for detecting multiple signals of a single disease;
c. more than two sensors for complex multi-signal profiles;
d. more than five sensors for comprehensive molecular profiling; and
e. more than ten sensors for multiplexed analysis covering heterogeneous disease states.

8. The method of claim 1, wherein the diagnostic kit is configured to detect disease-specific signal panels for:
a. neurological disorders, including Alzheimer's disease, Parkinson's disease, amyotrophic lateral sclerosis (ALS), multiple sclerosis, epilepsy, and stroke;
b. cancers, including breast, lung, colorectal, pancreatic, prostate, ovarian, gastric, liver, kidney, bladder, thyroid, lymphoma, leukemia, and melanoma;
c. metabolic disorders, including diabetes mellitus (type 1 and type 2);
d. autoimmune diseases, including systemic lupus erythematosus and rheumatoid arthritis; and
e. infectious diseases, including tuberculosis and hepatitis.

9. The method of claim 1, wherein the diagnostic kit integrates multiple body fluids for unified signal detection, including:
a. a combination of urine and saliva for complementary signal analysis;
b. tandem analysis of urine and exhaled breath for signals; and
c. combined analysis of urine, sweat, and tears for multi-systemic diagnostic insights.

10. The method of claim 1, wherein the system is further configured for specific patient groups, including:
a. routine screening of asymptomatic individuals for early disease detection;
b. monitoring of individuals at increased risk due to genetic predisposition, environmental factors, or lifestyle behaviors;
c. diagnosis of diseases in individuals presenting with non-specific symptoms; and
d. monitoring disease progression and therapeutic response in patients with established diagnoses.

11. The method of claim 1, wherein signal collection intervals are adapted based on the dynamics of signal excretion, comprising:
a. intervals of at least one hour, two hours, five hours, ten hours, fifteen hours, twenty hours, and up to twenty-four hours or more.

12. The method of claim 1, wherein the reader incorporates an AI-enabled system for:
a. predictive modeling of disease progression;
b. integration with electronic health records (EHRs) to support clinical decision-making; and
c. generating therapeutic recommendations based on diagnostic outputs.

13. The method of claim 1, wherein the system facilitates therapeutic delivery to metastatic sites by:
a. detecting metastatic signals and delivering therapeutic agents to secondary tumor sites; and
b. acting as a therapeutic to neutralize metastatic cancer cells directly.

14. The method of claim 1, wherein the system combines diagnostic and therapeutic functionalities, allowing simultaneous signal detection and targeted therapeutic delivery for integrated disease management.

15. The method of claim 1, wherein the system is adaptable for multiplexed detection of signals spanning multiple diseases, facilitating comprehensive diagnostic and therapeutic applications.
